(19) United States
(12) Patent Application Publication
(10) Pub. No.: US 2011/0206768 A1
(43) Pub. Date: Aug. 25, 2011

(54) MEDICAL PREPARATION

(75) Inventors: Dougal Frazer Laird, Hamilton East (NZ); Michael Roger Mucalo, Ohaupo (NZ); Subasinghe Nisanke George Premalal Jayantha Dias, Waverley (NZ)

(73) Assignee: The University of Waikato, Hamilton (NZ)

(21) Appl. No.: 12/517,755

(22) PCT Filed: Dec. 6, 2007
(86) PCT No.: PCT/NZ07/00354

§ 371 Date: Jan. 8, 2010

(30) Foreign Application Priority Data
Dec. 8, 2006 (NZ) 551965
Jan. 10, 2007 (NZ) 552568

(51) Int. Cl. A61K 35/32; A61K 9/00; A61P 19/08; B05D 7/00; B05D 3/02; B05D 3/10

(52) U.S. Cl. 424/484; 424/549; 427/2.26

(57) ABSTRACT

A bone matrix, including: a bone matrix material, which has had organic material removed, and a replacement material that has replaced the organic material, the bone matrix characterised in that the bone matrix is formed from a single piece of bone.

### TECHNICAL FIELD

This invention relates to a medical preparation, and methods of manufacturing same.

### BACKGROUND ART

In particular, the present invention relates to the preparation of bone material for implantation. It should be appreciated that the ultimate goal of using bone grafts or a bone substitute is to initiate a healing response that will produce new bone as an end product in an area where new bone is required.

The present specification will now be written with reference to the bone implants as being in relation to cancellous bone which is the internal spongy bone. In contrast, cortical bone makes up a large proportion of the skeletal mass, and is the structural bone.

Bone implants impregnated with titanium (and/or other metals such as aluminium and vanadium) have previously been used to replace cortical bone deficits or provide a structural support to allow healing.

However, there are significant concerns about the toxicity of these implants, particularly if an analogue is used in relation to cancellous bones which fit into the interior of a cortical bone.

When cancellous or cortical bone becomes damaged or lost, it is common to use a bone graft as part of the surgical repair the bone or bone deficit. Correct porosity and internal pore architecture have been shown to greatly enhance the healing, regenerative processes within the body. This is why it is desired to use bone based implants with similar architecture, rather than synthetic equivalents.

One procedure is the use of autogenous bone which is bone taken from the patient's own body. With this option, patients usually undergo two separate surgical procedures—one to remove the healthy bone and then another to implant it into the damaged area.

Autogenous bone is an ideal implant as it is highly compatible with the patient, is a long term match and has the required strength and biological functions. In particular the advantages include:
Superior osteogenic capacity
Contributes cells capable of immediate bone formation
Allows for bone induction by recipient bed where nonosseous tissue is influenced to change its cellular function and become osteogenic
Lack of histocompatibility differences or immunologic problems
Ease of incorporation
Lack of disease transmission
Autogenous cancellous bone has osteogenic, osteoinductive, and osteoconductive properties owing to the surviving bone cells, collagen, mineral, and matrix proteins, as well as a large trabecular surface area that is joined together as new bone forms.

This procedure however has a number of limitations such as
Additional incision or wider exposure, prolonged operative time, and increased blood loss and trauma
Increased postoperative morbidity from pain and potential infection or deformity
Sacrifice of normal structure and weakening of donor bone
Risks of significant complications
Limitations in size, shape, quantity, and quality (the supply is limited, especially in children)

Another option is the use of cadaver bones, these are bones which have been harvested from other humans who have died and chosen to donate their bodies tissues and organs. Unfortunately, with these bones there is concern about the product's supply and purity. For example, it may be possible for the surgeons or donor banks to overlook potential diseases within the bone, such as HIV, cancer and hepatitis, which may then be passed onto the recipient. Also, patients can have psychological issues or religious issues with using bones from this source.

There are a wide range of synthetic materials available in the market however few have physical properties (similar to human bone), zero disease risk, biocompatibility, osteo-conduction combined with the bone regeneration (osteo-induction) possibilities available through this process.

One example of a synthetic product is Vitoss® blocks. These are made from β tri-calcium phosphate which is a highly soluble material in vitro which is not desirable. While this material has its uses, it does not have the same structural integrity as does natural bone. Further, these products are very expensive to produce in comparison to natural bone.

Other examples of synthetic products include Cerabone® and Endobon® implants. These do not undergo the same degradation as Vitoss® and have the advantage of retaining the interconnecting pore structure of original bone. However, these still have disadvantages when used as bone implants, for example there is no biological function provided such as oestoinduction provided by autogenous bone implants.

Another attempt to find a suitable cancellous bone implant is described in PCT/GB1989/01020. This patent specification describes a process by which bovine bones are ground up and then mixed with gelatin to create an implant. This product however does not retain the internal architecture of natural bone which enhances integration, healing and regenerative processes within the body.

Further the steps of grinding the bone and then re-assembling the ground bone into an implant is a time consuming and expensive process.

In addition, the use of multiple bone pieces means that the implant has less strength, is harder to integrate into the body, and is more difficult to manufacture.

The use of gelatin is also of concern as it is not necessarily a prion-free material.

Having prion-free material is of considerable importance given the incidence of BSE. This is one reason why a ready source of natural bone material (bovine) has not been adopted widely despite their abundance as a ‘waste product’ from the meat industry. If bovine (and perhaps other animals) material could be used, then this could solve a number of problems in the prior art, namely finding material which is sufficiently bone-like to give the sufficient strength, architecture and integration in the body.

European Patent No. 1338291 uses bovine and porcine bones in an attempt to produce a bone matrix used in implants. However, like the invention disclosed in PCT/GB1989/01020 this relies on the grinding of the bone to form a sponge and mixing this with gelatin to form a sponge mass that can be implanted. Again, this does not have the same structure or strength as natural bone.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a bone matrix including

a bone matrix material, which has had organic material removed, and

a replacement material that has replaced the organic material

characterised in that the bone matrix material is formed from a single piece of bone.

According to one aspect of the present invention there is provided a method of producing a bone matrix including the following steps.
a) removing the original organic material from a single piece of bone matrix material, and
b) infiltrating the processed bone matrix material with a solution of replacement material,
the method characterised by the step of:
c) curing the replacement material inside the processed bone material.

In a preferred embodiment the bone matrix of the present invention may be used as a bone implant, and shall be referred to as such herein.

However, one skilled in the art would readily realize that the bone matrix of the present invention could also be used for other purposes, for example the bone matrix could be used for filling voids (or augmentation) in other structures.

Also, while not bone related, the material could be shaped to use as an ocular implant.

In a preferred embodiment the bone matrix material may be of a type that can be used to replace cancellous bone within the body. However, one skilled in the art would realise that the present invention may also have uses in relation to replacing cortical bone in some instances.

In a preferred embodiment the bone matrix material may be natural bone, and shall be referred to as such herein.

It should be appreciated that the bone matrix material preserves through its original process, numerous macropores and interconnected canals between macroporous cavities, which allows that a rapid colonization of cells at the center of the biomaterial.

An adapted porous structure is necessary to obtain a bone substitute capable to be degraded by bone cells and so to be replaced by new bone.

In a preferred embodiment the bone matrix material may be cancellous bone.

In a preferred embodiment the bone matrix material may have a bulk density of substantially, or greater than 0.8 g/cm³ (as measured by gravimetric (weighing of cube) analysis together with the physical dimensions of the cubes).

Below this level the bulk density value the inventors have found that the sintered bone has little intrinsic mechanical strength.

In a preferred embodiment the bone matrix material may be bone sourced from a variety of sources, including porcine, cervine, ovine and human cadavers.

In a particularly preferred embodiment the bone matrix material may be sourced from bovine, and shall be referred to as such herein.

One reason for using bone matrix material from a bovine source is that there is a plentiful supply, especially from abattoirs and animal/meat processing plants.

Another significant advantage of using bone sourced from bovine is that a key aspect of the present invention is the use of a single bone piece to produce an implant.

Bovine bone is of sufficient size that samples can be cut therefrom, treated in accordance of the present invention and then cut to size and shape without requiring more than one bone piece to do so. Further, any left over treated bone can be used as void filler.

One skilled in the art would appreciate that the source bone may be cut and shaped to the desired implant size and shape either before or after processing to remove organic material, and replace same with a replacement material.

However, this should not be seen as limiting, as a range of other bone matrix material may also be utilised with the present invention, for example from horses or other larger animals.

It should be appreciated that some bone implants may not be large, and therefore, sourcing the bone matrix material from other animal sources may be possible.

A significant problem with bovine bone is that in many countries there have been outbreaks of BSE (Bovine Spongiform Encephalopathy) otherwise known as Mad Cow Disease. This can be introduced to humans through the ingestion or introduction to the body of proteins known as prions obtained from beef products. The human form of this disease is known as Creutzfeldt-Jakob Disease (CJD).

In preferred embodiments of the invention the bone material is sourced from certified BSE free countries such as New Zealand and Australia.

Bovine bone sourced from New Zealand is BSE free. Bovine bone sourced from a certified BSE free country is an ostensibly cheaper material due to the heavy agricultural practices and high volumes of bovine bone being the waste product of abattoirs and meat processing plants.

Bovine bone is normally used as a low grade fertilizer. Alternatively, the bone must be disposed of into the environment.

Bovine bone from certified BSE free countries can be sourced from mainstream herds and meat processing plants. This is a significant advantage, as there is no requirement to develop a ‘safe’ herd of cattle to supply clean BSE free bone.

If bone is sourced from non BSE free countries, for example Europe or even the USA, a number of disadvantages are present, these include the following:
Far more care would be required in sourcing the bone material to ensure that it is safe for use. This would significantly increase the cost, for the time and labour required, as well as compliance costs for safety.
More care and quality control would be required during the step of removing the organic material from the bone matrix material. Although this step should ensure that the bone material is clean and prion free, if bone is sourced from a country known to have BSE, more rigorous procedures, testing and quality control would be required. Again, this would increase the manufacturing cost significantly.

The use of waste bone material also provides advantages in lower disposal costs and lower volumes of waste having to be disposed of.

Regardless of the bone source, (human, bovine or whatever) it has been recognised by the inventor that all of the organic material within the bone should be removed, not only to allay any concerns about the material containing prions, but also to remove other potential diseases such as HIV, cancer and hepatitis.

The organic material can be removed by a number of ways.

In one embodiment of the present invention the bone may be subjected to heat and pressure in an aqueous medium to remove the bulk of the protein in lipid organic material.

In a preferred embodiment the bone (as processed with heat and pressure) may then be sintered to burn off any remnant organic material.

The inventor has found that this process effectively sterilises the bone to guarantee a product completely free of prions and any other diseases.

It should be appreciated that this is a preferred method of organic material removal only.

One skilled in the art would readily appreciate that other methods may be used to remove organic material from the bone matrix material. These may include, for example treatment with a chemical reagent or solvent (such as, sodium hydroxide, hydrogen peroxide, or acetone), although it is thought that these alone would not provide the degree of sterilisation required.

It has been recognised that the inclusion of a bone matrix material with the organic matter removed is often chalky in consistency and correspondingly is of low strength. Collagen which forms part of the organic material removed strengthens the bone matrix and provides a framework for bone growth.

While the bone (with organic material removed) has the requisite pore architecture it does not have the internal material which allows for the ready integration of the bone implant with the physiology of the body into which the implant is being placed.

Therefore, the present invention includes the replacement of the original organic material with a replacement material.

Ideally the replacement material is one that has properties that to a degree mimic the functionality of autogenous gone. For example the replacement material may impart strength to the bone matrix and act as a reinforcing material. Alternatively, or additionally, the replacement material may supply biological functionality similar to that of autogenous bone.

This is because bone grafts serve a dual mechanical and biological function. Initially, some mechanical strength is important—mainly for handling, however, it is ultimately the biological activity that allows for incorporation into the host bone.

A variety of replacement materials can be utilised with the present invention. These include synthetic calcium phosphates (such as β-tri-calcium phosphate), synthetic hydroxyapatite or even algae or coral-derived hydroxyapatite.

Calcium phosphate (Hydroxyapatite) derived materials are known to be biocompatible and capable of bonding chemically to bone. They are widely used as bone repair materials in human surgery because their chemical composition is similar to that of bone. This is a preferred replacement material.

Calcium phosphate apatite (CPA) is known to be one of the most important implantable materials due to its biocompatibility. Natural bone is approximately 70% CPA including hydroxyapatite (HAP) by weight and 50% by volume

In preferred embodiments the replacement material may be an organic material based matrix as it has a number of advantages over synthetic derived products. These include the following:

Firstly, an organic matrix is less likely to have toxicity problems.

Secondly, an organic matrix provides additional strength to the bone structure.

Further, an organic material may enable, enhance or initiate integration of the bone into the transplantee's body. That is, it is believed the body could break down the organic structure and replace it with collagen fibres, further strengthening the position of the bone implant in the body and its overall strength.

There is a wide variety of suitable organic matrix materials that can be used with the present invention. These include (but are not limited to):
keratin which is derived from wool,
glucosamine, which can be sourced from shellfish, cartilage (such as shark skeletons), and in some cases corn,
chondroitin which is sourced from pure animal cartilage, or
gelatine which is also animal derived.
bone marrow aspirate (preferably autogenous)

It should be appreciated that one problem with using animal sourced replacement materials is that there may be a risk of reintroducing some of the diseases that were removed by removing the original organic material from the bone matrix material in the first place. This is especially the case when the replacement material is derived from a land based animal. This must be taken into account and sufficient testing put in place to ensure sterile material is used, if the reinforcing material is sourced from animals. Rigorous treatment of the reinforcing material to ensure that they are sterile may increase the cost of manufacture and of the bone matrix product.

Another concern in using animal sourced reinforcing material, for example shark sourced materials, is that these could have toxicity problems associated with the high level of heavy metals they contain, which could then be introduced to the patient.

In another embodiment the reinforcing material may be derived from plants, for example oxycellulose.

In a preferred embodiment the replacement material may also be bio-active or contain bio-active materials. That is, the replacement material could initiate or enhance integration of the bone matrix into the patient's body. This significantly decreases recovery time, and increases the strength and integration of the implant.

For example the replacement material may have within it a number of bioactive components that help the bone integrate more readily into the body and/or encourage bone healing such as bone morphogenic proteins, bone and growth hormones or autogenous cells such as cells sourced from the recipient's bone marrow.

In one particularly preferred embodiment the preferred organic material may be chitosan which is derived from shellfish shells.

Chitosan is particularly suitable for the present invention as some believe it is a bio-active that the body can break down and replace with collagen fibres.

In another embodiment the replacement material may be synthetic. One example of a suitable synthetic material is polycaprolactone (PCL) which is a biodegradable thermoplastic polymer derived from chemical synthesis of crude oil. Even though this material is synthetic, it has the advantage that it has already been used instead of titanium in repairing holes is skulls, thus is known to be bone compatible.

In another alternative embodiment, the replacement material may be a combination of above mentioned components, or a combination of one (or more) of these with other bio-compatible components.

An important aspect of the present invention is the use of a single bone piece to manufacture the bone matrix (implant). This is because of its associated strength, bio-compatibility and processing properties.

However, there are inherent problems with using larger pieces of bone which the inventors have needed to overcome in order to implement the present invention.

A significant problem having larger bone than used in the prior art (which generally utilises ground up bone) is that the replacement material needs to be impregnated throughout the natural bone structure.

This is relatively easily achieved with ground pieces of bone utilised in the prior art as a consequence of the natural diffusion process and the greater accessibility of pores.

However, the longer distances that the replacement material has to travel within larger bone pieces, and through the natural bone structure makes it difficult to achieve the full penetration and strength required and also makes rinsing the entire bone of impurities a challenge.

One way to address this problem is to lower the viscosity of the replacement material so that it flows through the porous bone structure more readily. However, this can also lead to decreased strength and the replacement material pouring out of the bone as well.

Therefore, according to one aspect of the present invention there is providing the step of curing the reinforcing material within the bone.

The term “curing” as used herein should be taken to mean any process that ensures that the replacement material can set within the bone.

Thus, the ‘curing’ process is most likely a process that causes the replacement material to change its viscosity after it has infiltrated the porous structure of the bone.

The curing process may be achieved by a variety of means depending on the material being infused.

For example, in one embodiment a vacuum process may be used to draw the replacement material through the bone sufficiently quickly that the material penetrates the bone before it has time to set/cure.

Setting of the replacement material, for example could be the result of evaporation, of a solvent, the action of a curative agent, and/or application of heat.

It is apparent that the present invention has a number of advantages over the prior art, these include:
Avoiding the need to use autogenous bone and its associated difficulties such as chronic, often debilitating pain from the harvesting operation, blood loss, chance of infection, and longer hospital stay and recovery time. The second surgery also adds substantially to the financial cost the present invention does not require a second surgical site.
Maintaining the correct porosity and internal pore architecture required to enhance healing and the regenerative processes within the body. The required tissue architecture for tissue in-growth into the bone matrix is also present which removes the necessity to generate it via an artificial process.
Allografts eliminate the need for a second surgery, however the grafted bone may be incompatible with the host bone and ultimately rejected. Allograft also poses a slight but troubling risk of introducing a variety of viruses in the patient, including AIDS or hepatitis. Allografts could be treated with the present invention to ensure a sterile product. Bone Matrix sourced from cow bone is also sterile.
Use in at least the following applications
   Defect filling in total hip revision
   Spinal fusion
   Hand and foot surgery
   Simple and complex fractures repair
   Joint reconstruction
   Non-union or pseudarthrosis, arthrodesis and osteotomies
   Prosthesis revision surgery
   Spinal fusion
   Cyst treatment
   Limb salvage
Less processing time as the single bone does not need to be broken down and then reassembled.
A disease free implant. The bone matrix of the present invention can be guaranteed pathogen and prion free which is a desirable and marketable attribute for end-users receiving the implant.
Required strength and integration within the transplantee's body.
A process that cures the material within the bone enables larger bone pieces to be used with all of the advantages as described above.
In contrast to unmodified sintered bone products, there is some putative strengthening brought about by the infiltration of the reinforcing material into the bone matrix structure.
Bovine bone is readily and relatively cheaply sourced in New Zealand from the mainstream cattle population which is BSE-free.
The bone matrix is processed (i.e. sintered) such that any harmful proteinaceous components have been burnt out so minimising the risk of transmission of prions to zero.
The organic material is replaced with a naturally sourced material (for example chitosan). Therefore the bone matrix is not re-infiltrated with bovine or human or other CJD-dementia-prone mammal's collagen (a potential source of prions).
Enhancement of integration of the replacement bone through use of bio-active components.
The process is therefore readily employable anywhere in the world, even if prion-ridden bovine bones were used.
The process does not require the development of controlled herds so the price of the source material is cheaper.
The bone matrix can be easily shaped into the desired shape. This is due to the bone matrix material being a single piece of bone that is sintered and chalky (at the initial stage of its processing) which can be shaped to a desired geometry and then infiltrated.
The process of the present invention, utilises material, for both the bone matrix material and the reinforcing material by-products which would otherwise enter landfills (i.e. waste abattoir and crustacean hard tissue).
The infiltration material can be varied to include medications to help bone healing (e.g. bone morphogenetic proteins).

### BRIEF DESCRIPTION OF DRAWINGS

Further aspects of the present invention will become apparent from the following description which is given by way of example only and with reference to the accompanying drawings in which:

FIG. 1 shows bone implant samples of sintered cancellous bone (SCBB), and SCBB infiltrated with organic reinforcing;

FIG. 2 shows a stress versus strain graph of raw bone, reinforced sintered bone and sintered bone,

FIG. 3 shows the average total number of moles of ammonaiacal N in samples after hours of washing in distilled water. Control samples showed no presence of ammonaiacal N, and

FIG. 4 shows the average number of moles of Cl— in samples after hours of washing in distilled water, and

FIG. 5 shows the relationship between Log₁₀ Toughness versus Log₁₀ Apparent Density

### BEST MODES FOR CARRYING OUT THE INVENTION

An ideal bone matrix graft material includes the following characteristics many of which are physical and have been characterised by competing products.

Composition (Ratio of Hydroxyapatite and Chitosan)
Advantage: Control of bone substitution rate.
Benefit: Newly formed bone with the same quality as natural bone, resorption kinetic adapted for human bone remodelling.
Porosity %
Advantage: Balance between bone in-growth and mechanical properties.
Benefit: Rapid bone in-growth, easy use of materials.
Macroporosity %
Advantage: Bone cells colonization at the centre of biomaterials.
Benefit: Homogeneous bone in-growth in the materials.
Microporosity
Advantage: Diffusion of biological fluids in substitute.
Benefit: Allows diffusion of nutrients of the materials, evacuation of cellular wastes, favour ionic exchanges (dissolution-precipitation).
Interconnection
Advantage: Rapid cellular colonization at the centre of biomaterial.
Benefit: Rapid bone in-growth.
Compressive strength: >10 MPa
Advantage: Mechanical properties.
Benefit: Easy use of materials by surgeon. Stability of substitute.

One method of trying to achieve this is described below.

Cancellous bovine bone is acquired from the New Zealand meat industry (known to be prion free) as a waste feed stock.

Samples of bone are cut to a desired shape and size, and then subjected to heat and pressure in an aqueous medium to remove the bulk of the protein and lipid organic material.

The processed bone is subsequently sintered at 1000° C. for three hours to burn off remnant organic material, and as a means of sterilisation (by incineration) to guarantee a product completely free of prion material.

A chalky bone results of low strength.

### EXAMPLE ONE

A replacement material is prepared by dissolving 2.5 g of chitosan and 1.5 g CaHPO₄ in 250 mL of water acidified with HCl to yield a 1% w/v solution of chitosan and a 0.6% w/v solution of calcium hydrogen phosphate.

This solution with viscosity marginally greater that that of water is infiltrated into the porous sintered cancellous bovine bone (SCBB) by placing the SCBB in a vacuum capable vessel and then inducing a vacuum. Once a sufficient vacuum has been reached, the line to the vacuum is shut off and a line to the infiltrate is opened allowing the liquid to flow into the sintered bone. The chitosan solution is forced into the SCBB pores and also into the microscopic crevasses within the SCBB's structural trabeculae.

After infiltration the sample is removed and placed in an atmosphere of NH_{3(g)} as per the curing regime outlined below and then washed as per washing process outlined below.

The sample is air dried to yield a mechanically strengthened bone replacement bio-implant that is biocompatible and bio-absorbable while retaining comparable pore size and internal pore architecture to that of living bone tissue.

Correct porosity and internal pore architecture have been shown to greatly enhance the healing, regenerative processes within the body.

The chitosan infiltrating material on its own has been shown not to elicit antigenic responses when placed in the body, so making it an ideal candidate as a bone replacement biomaterial [11].

Although the yield strengths under compressive strain of these chitosan infiltrated SCBB samples tend to be less than that of raw bone, their subsequent compressive stress versus strain profiles are characteristic of bone.

The degree of infiltration was established by Scanning Electron Microscopy, Dye Tracing and InfraRed Microscopy.

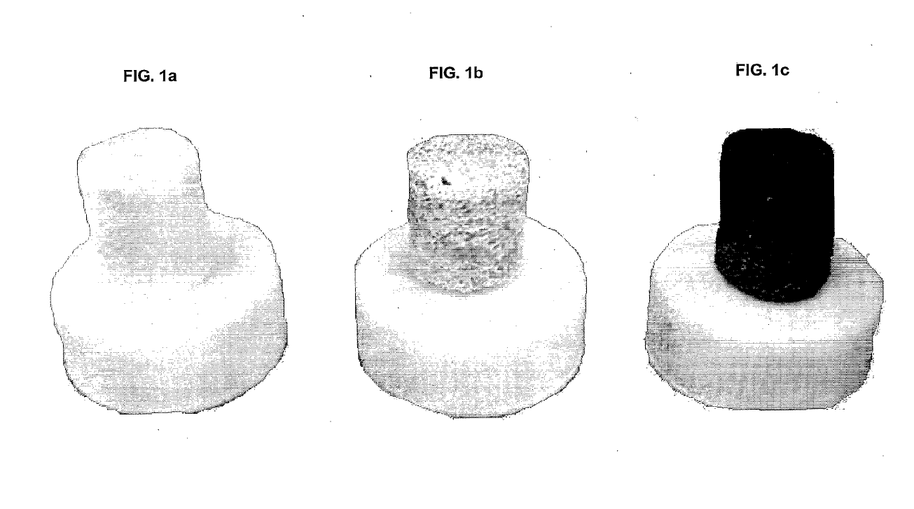

FIG. 1 shows SCBB and two infiltrated sintered bone samples.

Mechanical strength was tested by compression testing on an Instron instrument. Comparison between pure sintered bone, raw bone and treated samples were made with regard to yield strength, Young's modulus and stress/strain profile. Due to the inherent natural variability in structure and density of the raw material (SCBB) a statistical approach was needed to make a comparison between this material and the infiltrated reinforced samples.

This statistical analysis was undertaken in a series of steps. First, compression testing was performed on a number of untreated SCBB samples. ‘Apparent’ density was calculated simply by dividing the mass of the sample by the volume. From this data, and the compression testing data, a correlation plot of maximum stress versus apparent density was produced.

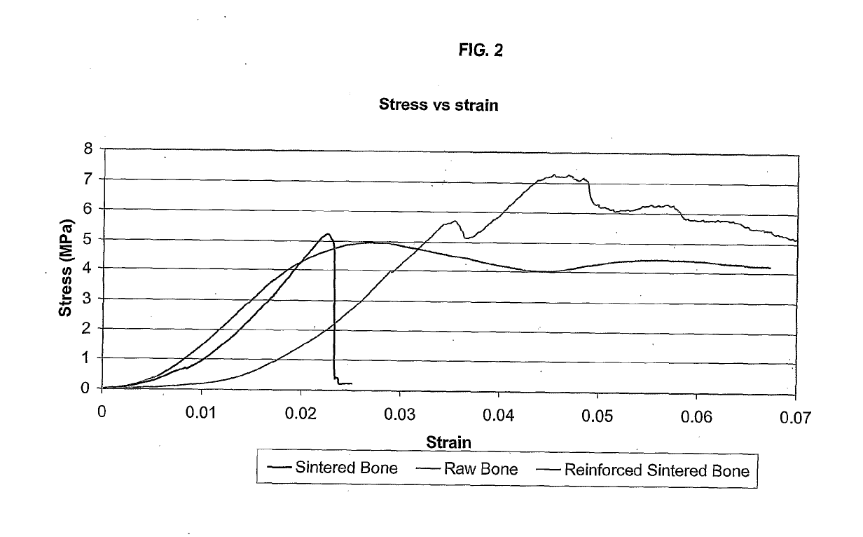

Subsequent reinforced samples' test results were compared to this baseline value as a means of gauging improvements or otherwise to physical properties. Initial results indicated that some organic matrices led to slightly higher maximum stress values. Significantly the stress/strain profiles began to resemble those of raw cancellous bone rather than those of sintered bone as shown in FIG. 2. The profile showed that after stress induced failure, the reinforced sintered bone still retained some mechanical strength. This contrasts markedly to sintered bone which shows a complete lack of mechanical strength after failure.

### EXAMPLE TWO

Other SCBB samples are made via similar processes to those described above but without the ‘curing’ process using NaOH solution described above.

Polycaprolactone (PCL) is dissolved in tetrahydrofuran (THF) and infiltrated into SCBB. The THF solvent is then allowed to evaporate to yield a reinforced sintered bone sample. PCL is a biocompatible polymer that is broken down in the body to non-toxic by-products.

Other strengthening materials such as oxycellulose and keratin derived from wool fibre can be in principle infiltrated into the SCBB by the processes described above.

Ultimately, a cheap, widely available raw material (waste bovine bone) is transformed into a highly biologically compatible bone replacement bio-implant material, with superior mechanical properties to that of unmodified sintered bone and without the risk of any bone-sourced prion transmission.

Biocompatibility

Sintered bovine bone has previously been shown to be biocompatible in vivo as shown by reference [1] in which sintered bone has been successfully used in spinal surgery.

Additionally, bovine cancellous bone, that has not been sintered but has been deproteinated has been shown to be osteoconductive in a sheep and a dog model [2, 3].

Furthermore, reinforcing materials such as polycaprolactone, chitosan and silica which can be utilised with the present invention have been shown to be individually biocompatible (or assisting biocompatibility when used in a hybrid material) on the basis of previous reports [4-6]

Range of Reinforcing Materials

The following replacement materials were utilised in experimentation on curing processing.
Chitosan
Chitosan+Calcium Phosphate (co-precipitated)
Chitosan+TEOS
Chitosan+genipin
Chitosan+genipin+calcium phosphate (co-precipitated)
Chitosan+Titanium tetraisopropoxide
Polycaprolactone

Curing Regime

Samples were cured by being placed in an atmosphere of NH_{3(g)} generated from concentrated NH_{3(aq)}.

The NH_{3(g)} diffuses and dissolves into the infiltrated solution occupying the pores of the bone matrix in which it hydrolyses to form hydroxide. The hydroxide then acts to cure the chitosan and accompanying materials when present.

For a sample of dimensions 12 mm diameter by 15 mm height this was seen to occur within 1 hour. Generally, samples were simply left overnight in a NH_{3(g)} atmosphere to ensure complete curing.

Washing Process

After curing, samples were washed in distilled water to remove excess ammonia and ammonium salt of the acid used to dissolve the chitosan and other compounds.

Washing was carried out by placing samples in a vessel with gently stirred water. 400 mL of distilled water is used per sample and the water was replaced every 1 hour.

It was found that 3 repetitions were necessary to reduce residue ammonia and other residues to levels either approaching control levels or where no further reduction in the levels of these impurities could be detected.

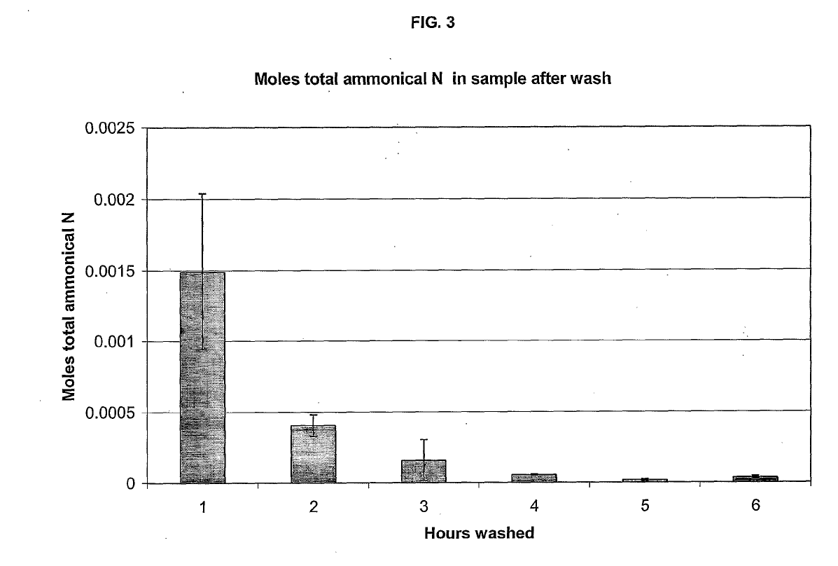

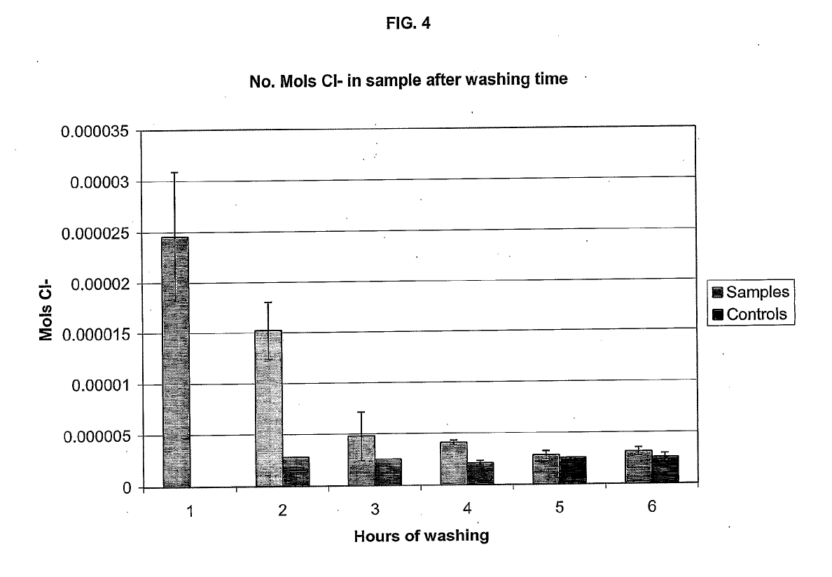

FIGS. 3 and 4 show the levels of ammonia/ammonium (total ammoniacal N) and chloride (due to the use of HCl to dissolve the chitosan) remaining in samples after the 1 hour washing cycles.

In particular FIG. 3 shows the average total number of moles of ammoniacal N in samples after hours of washing in distilled water. Control samples showed no presence of ammoniacal N, and

FIG. 4 shows the average number of moles of Cl— in samples after hours of washing in distilled water.

Preferred Properties of the Bone Matrix

The preferred properties of the bone matrix are cancellous bovine bone having a bulk density greater than 0.8 g/cm³ (as measured by gravimetric (weighing of cube) analysis together with the physical dimensions of the cubes).

Below this bulk density value sintered bone proved to have little intrinsic mechanical strength.

Bone Cleaning Methods

Autoclaving is recommended for 6 hours in water at 15 psi with a change of water every two hours. This would be followed by drying then sintering in a muffler furnace for 3 hours at 1000° C.

Mechanical Data

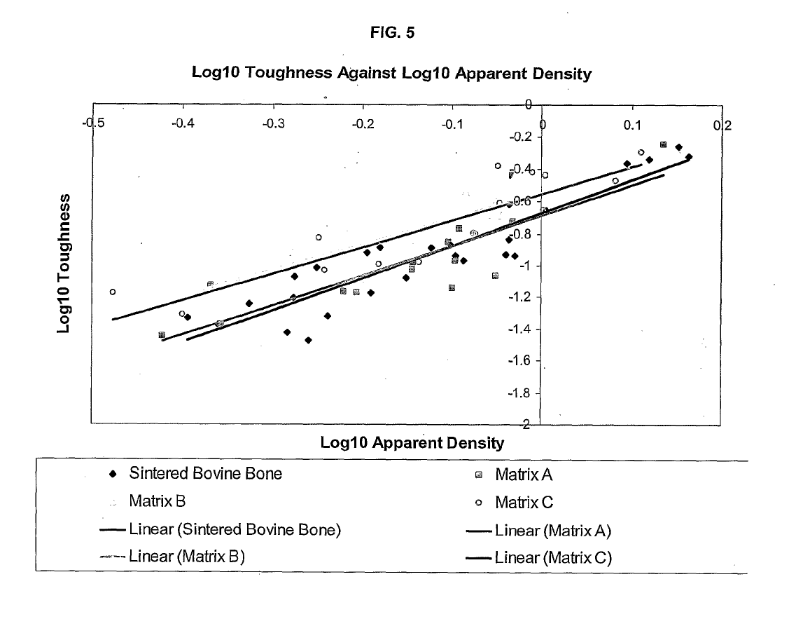

FIG. 5 shows that the infiltrated samples called Matrix B and C (including chitosan and chitosan/calcium phosphate respectively as the reinforcing material) demonstrate ostensibly greater Log₁₀ Toughness values than the equivalent uninfiltrated sintered bovine bone and polycaprolactone-infiltrated sintered bone.

In particular FIG. 5 shows the relationship between Log₁₀ Toughness versus Log₁₀ Apparent Density

### REFERENCES

1. Minamide, A., et al., The use of sintered bone in spinal surgery. European Spine Journal, 2001. 10(0): p. S185-S188.
2. Worth, A. J., et al., Combined xeno/auto-grafting of a benign osteolytic lesion in a dog, using a novel bovine cancellous bone biomaterial. Clinical Communication, In Press.
3. Worth, A., et al., The evaluation of processed cancellous bovine bone as a bone graft substitute. Clinical Oral Implant Research, 2005. 16: p. 379-386.
4. VandeVord, P. J., et al., Evaluation of the biocompatibility of a chitosan scaffold in mice. Journal of Biomedical Materials Research, 2002. 59(3): p. 585-590.
5. Gough, J. E., et al., Craniofacial osteoblast responses to polycaprolactone produced using a novel boron polymerisation technique and potassium fluoride post-treatment. Biomaterials, 2003. 24(27): p. 4905-4912.
6. Koo, H.-J., et al., Antiinflammatory effects of genipin, an active principle of gardenia. European journal of pharmacology 2004. 495(2-3): p. 201-208.
7. Madihally, S. V. and H. W. T. Matthew (1999). “Porous chitosan scaffolds for tissue engineering.” Biomaterials 20: 1133-1142.

Aspects of the present invention have been described by way of example only and it should be appreciated that modifications and additions may be made thereto without departing from the scope thereof of the appended claims.

1. A bone matrix, comprising: a bone matrix material, which has had its indigenous organic material removed, and a replacement material that has replaced the indigenous organic material, characterised in that the material is immobilized within the bone matrix material by precipitation of the replacement material by adjusting pH and that the bone matrix is formed from a single piece of bone.

2. A bone matrix as claimed in claim 1 wherein the bone matrix material is of a type that can be used to replace cancellous bone.

3. A bone matrix as claimed in claim 1 wherein the bone matrix material is natural bone.

4. A bone matrix as claimed in claim 1 wherein the bone matrix material is bovine bone.

5. A bone matrix as claimed in claim 1 wherein the bone matrix material has a bulk density of substantially 0.8 g/cm³ or greater.

6. A bone matrix as claimed in claim 1 wherein the bone matrix has a compressive strength of substantially 10 MPa or greater.

7. A bone matrix as claimed in claim 1 wherein the replacement material comprises a component selected from the group consisting of synthetic calcium phosphates, polycaprolactone, synthetic hydroxyapatite, algae-derived hydroxyapatite, coral-derived hydroxyapatite, keratin, glucosamine, cartilage, chondroitin, gelatine, oxycellulose, chitosan, bone marrow aspirate, and bone growth hormones.

8. A bone matrix as claimed in claim 1 wherein the replacement material is an organic material based matrix.

9. A bone matrix as claimed in claim 8 wherein the replacement material comprises a component selected from the group consisting of keratin, glucosamine, cartilage, chondroitin, gelatine, oxycellulose, chitosan, bone marrow aspirate, bone growth hormones and bone morphogenic proteins.

10. A bone matrix as claimed in claim 1 wherein the replacement material is impregnated throughout said single piece of bone.

11.-16. (canceled)

17. A bone matrix as claimed in claim 7, wherein the replacement material comprises calcium phosphate and chitosan.

18. A bone matrix as claimed in claim 1, wherein the single piece of bone is sourced from a certified BSE-free source.

19. A method of producing a bone matrix, comprising:
a) removing the indigenous organic material from a single piece of bone matrix material to form a processed bone matrix material,
b) infiltrating the processed bone matrix material with a replacement material, and
c) immobilizing the replacement material inside the processed bone material by precipitating the replacement material by adjusting the pH.

20. A method as claimed in claim 19, wherein the removing the indigenous organic material comprises heat and pressure treatment in an aqueous medium.

21. A method as claimed in claim 19, wherein the removing the indigenous organic material comprises sintering.

22. A method as claimed in claim 19, wherein the infiltrating the processed bone matrix material with a replacement material comprises use of a vacuum.

23. A method as claimed in claim 19, wherein the replacement material comprises chitosan and calcium phosphate, and the bone matrix has a compressive strength of substantially 10 MPa or greater.

24. A method as claimed in claim 19, wherein the adjusting of the pH of the replacement material comprises raising the pH.

25. A method as claimed in claim 19, wherein the single piece of bone matrix material is sourced from a certified BSE-free source.

26. A method of producing a bone matrix, comprising:
a) removing the indigenous organic material from a single piece of bone matrix material to form a processed bone matrix material, wherein the single piece of bone matrix material is sourced from a certified BSE-free source,
b) infiltrating the processed bone matrix material with a solution of replacement material, and
c) setting the replacement material inside the processed bone material.

27. A method as claimed in claim 26, wherein the setting the replacement material comprises an evaporation of a solvent, an action of a curative agent, or an application of heat.

28. A method as claimed in claim 26, wherein the removing the indigenous organic material comprises sintering.

29. A method as claimed in claim 26, wherein the infiltrating the processed bone matrix material with a replacement material comprises use of a vacuum.

30. A method as claimed in claim 26, wherein the replacement material comprises chitosan and calcium phosphate, and the bone matrix has a compressive strength of substantially 10 MPa or greater.
